(19) United States
(12) Patent Application Publication
(10) Pub. No.: US 2014/0360912 A1
(43) Pub. Date: Dec. 11, 2014

(54) LIQUID OR SEMISOLID COSMETIC COMPOSITION

(71) Applicant: COSMETIC WARRIORS LIMITED, Poole Dorset (GB)

(72) Inventors: Mark Constantine, Poole Dorset (GB); Margaret Joan Constantine, Poole Dorset (GB); Helen Elizabeth Ambrosen, Wimborne Dorset (GB); Rowena Jacqueline Bird, Ibsley Dorset (GB)

(73) Assignee: COSMETIC WARRIORS LIMITED, Poole Dorset (GB)

(21) Appl. No.: 14/372,612

(22) PCT Filed: Jan. 17, 2013
(86) PCT No.: PCT/GB2013/050099

§ 371 Date: Jul. 16, 2014

(30) Foreign Application Priority Data
Jan. 20, 2012 (GB) 1201162.3

(51) Int. Cl. A61K 8/92; A61Q 1/06; A61Q 1/04; A61K 8/02; A45D 40/00; A61Q 1/02

(52) U.S. Cl. CPC A61K 8/922; A45D 40/00; A61Q 1/02; A61Q 1/04; A61K 8/0216; A61Q 1/06; A61K 2800/30; A61K 2800/43; A61K 2800/591; A61K 2800/87
USPC 206/581; 206/525; 424/63; 424/64

(57) ABSTRACT

The invention relates to a base for a liquid cosmetic composition comprising a wax and a vegetable oil and to a liquid or semisolid cosmetic composition comprising the base and a pigment.

The present invention relates to a liquid or semisolid cosmetic composition and, in particular a liquid or semisolid lipstick composition formed from a liquid or semisolid cosmetic base. In addition, the invention relates to a container containing a liquid or semisolid cosmetic composition, for example a liquid or semisolid lipstick composition.

Lipsticks have been a long established method of applying coloured pigments to the skin. They are primarily applied to the lips but if the composition is adaptable enough, colour can be applied to the cheeks and even to the eye area.

In cosmetic products such as lipsticks, mineral oils and petroleum jelly have been commonly used as emollients for the skin. They are cost effective, easily available, and stable in compositions. They work by preventing the skin from losing moisture as they have an occlusive effect. Mineral oils have been used in cosmetics, colour cosmetics or make-up, for decades. They are used in cosmetic creams such as traditional “cold cream”, baby oils and the like.

When used in colour cosmetics and make-up, mineral oils and petroleum jelly are useful as they facilitate the dispersal of coloured pigments which deliver shades to decorate the skin on the face. In addition, their emollient nature helps to apply the colour, to adhere it to the skin and prevent the dryness of the pigments affecting the texture of the skin. The disadvantage of ingredients such as mineral oils and petroleum jelly is that they are sourced from the petrochemical industry. It is commonly known that the petrochemical industry adversely affects the environment and therefore it is desirable to limit the use of products derived from this source.

Mineral oil and similar ingredients can also have negative effects on the skin. Mineral oil often causes allergic reactions resulting in redness and itching. More seriously it can cause or aggravate acne rosacea, a complaint which leads to redness and pimples, particularly around the nose. This is very unpleasant to experience and distressing in appearance. Mineral oils have also been linked to skin cancer.

Vegetable oils are much more beneficial to the skin than mineral oils. They are not irritating and allow the skin to function in a healthy normal way and do not cause adverse effects. They soften the surface of the skin without the occlusive effect of mineral oils.

Vegetable oils have been used in cosmetic products for centuries and were the basis for the first cold cream. This formula is thousands of years old, having been invented by the Greek physician Galen in 150 AD. It was the first cosmetic cream and consisted of beeswax and olive oil with water (or rose water) beaten into it.

Because it cooled the skin, it was dubbed “cold cream”. Over the centuries mineral oil has replaced vegetable oils because of its low cost. However this change to the use of mineral oil has not been advantageous to the skin and it would therefore be advantageous to the consumer to be provided with a cosmetic composition, particularly a lipstick, which contains vegetable oils rather than mineral oils or petroleum jelly.

The method of manufacture of lipsticks is well known and the resultant sticks of colour are iconic and well recognised. However the production of the stick requires careful moulding techniques which are time consuming, wasteful and therefore expensive.

A lipstick in liquid or semisolid form would therefore have the advantage that it can be filled into containers rather than moulded and can easily be applied with a brush or a pad.

The present invention seeks to overcome the disadvantages of known cosmetic compositions by providing a liquid or semisolid cosmetic composition which contains no mineral oils or petroleum jelly. In particular, the composition comprises a base to which a coloured pigment can be added to provide the finished product.

In a first aspect of the present invention, there is provided a coloured cosmetic composition for application to the lips or skin, wherein the cosmetic composition is liquid or semisolid at room temperature and comprises:
from about 10% to 50% by weight of a pigment; and
a cosmetic base;

wherein the cosmetic base is a liquid or semisolid at room temperature and comprises:
about 4% to 40% by weight of a wax; and
about 60% to 96% by weight of a vegetable oil.

Using vegetable oils and a natural wax, for example candelilla wax to form the cosmetic base, it was found that a stick composition could be produced which had the advantage of a positive environmental impact and which was, furthermore, non-irritating to the skin. It was further found that this hard product of a vegetable oil and a natural wax could be treated to give a soft and cream like texture. This negates the requirement for expensive, time consuming moulding techniques, to produce sticks and means that it could simply be filled into a suitable bottle.

Thus, this cosmetic composition overcomes the problems of the prior art as it is liquid or semisolid and does not contain mineral oils or petroleum jelly.

In the present application a “base” for a cosmetic composition (cosmetic base) refers to a composition to which coloured and/or perfumed ingredients may be added to obtain a cosmetic composition for application to the lips, skin or eyes. The base is therefore suitable for forming a solution or a homogeneous dispersion of a coloured pigment and/or a perfume.

In the present application, the term “liquid” refers to a composition which is in liquid form at room temperature, i.e. at a temperature of about 15° C. to 25° C., although the temperature range at which the product is liquid may, of course, be broader than this. Suitably, a liquid composition of the present invention should be sufficiently free flowing to be filled into containers by pouring but should be of sufficient viscosity to remain on the skin without running.

The term “semisolid” refers to a composition which, at room temperature, i.e. at a temperature of about 15° C. to 25° C., has the consistency of a cream, ointment or paste. Thus, a semisolid may not be free flowing in the same way as a liquid but it will not have the structural integrity of a solid and therefore will not retain a shape and cannot be moulded.

In one embodiment, the composition is a liquid and in an alternative embodiment it is a semisolid. Particularly suitable compositions are in semisolid form.

The cosmetic base is an oil based system. As it does not contain water there is no need for a cosmetic preservative such as methyl paraben or propyl paraben, which are commonly used preservatives in cosmetic compositions of colour cosmetics or make-up. They are powerful ingredients and can cause adverse effects on the skin. To provide a cosmetic product which does not require preservation is very desirable.

Suitable waxes are waxes which are cosmetically acceptable, i.e. which are suitable for use on the skin in that they do not irritate the skin, have an unpleasant odour etc. A person of skill in the art, such as a cosmetic chemist, would be aware of a large number of cosmetically acceptable waxes which could be used in the cosmetic base composition of the present invention. Examples of such waxes include candelilla wax, bees wax, Japan wax, carnauba wax, soy wax and palm wax or mixtures thereof, although other waxes may also be used. Candelilla wax, bees wax, Japan wax and mixtures thereof are particularly suitable.

Candelilla wax is a hard and brittle wax extracted from the wax-coated stems of candelilla shrubs (usually from the plant Euphorbia cerifera, syn. Euphorbia antisyphilitica). The plant grows wild in North-eastern Mexico and the plains and foothills of the Chihuahua desert. Commercial uses for candelilla wax include polishes, candles, lubricants, paper waterproofing and cosmetics. It is often used to replace other waxes, to add texture, or to help make barrier products, such as lip balms. Compatible with most waxes and a variety of other ingredients, it is a versatile substance.

Bees wax is obtained from the hive of honey bees. It is well known for use in candles, cosmetics and polishes.

Japan wax is a pale yellow waxy water insoluble solid obtained from the fruit of trees of the genus Rhus, for example Rhus Succedanea or Rhus verniciflua. It is widely used in cosmetics, candles and polishes.

All of the waxes mentioned are well known and widely available from a number of suppliers.

In particularly suitable compositions, the wax comprises candelilla wax, optionally in admixture with one or more other waxes, for example bees wax or Japan wax. In certain suitable compositions, the wax consists of candelilla wax.

As with the wax, suitable vegetable oils will also be cosmetically acceptable and a skilled cosmetic chemist would be aware of a large number of suitable oils which could be employed. Examples of such vegetable oils include jojoba oil, olive oil, sweet almond oil, castor oil, avocado oil, rosehip oil and argan oil or mixtures of two or more of these. Jojoba oil, olive oil, sweet almond oil, castor oil and avocado oil are particularly suitable. Many other cosmetically acceptable vegetable oils are known and could also be used in the compositions of the invention in addition to or in place of those mentioned above.

These ingredients do not have the environmental disadvantages of mineral oil and indeed they are available as certified organic materials. Like the waxes mentioned above, they are also well known products which are available from a wide range of suppliers.

More suitably, the cosmetic base composition of the invention comprises from about 10% to 35% by weight of a wax and about 65% to 90% by weight of a vegetable oil.

Advantageously, the cosmetic base composition comprises from about 15% to 25% by weight of a wax and about 75% to 85% by weight of a vegetable oil.

The amount of pigment used in the coloured cosmetic composition will vary according to the depth of colour required. The cosmetic base of the present invention has proved to be particularly useful in the manufacture of liquid or semisolid lipsticks as it capable of dispersing a large amount of pigment. In the past, liquid or semisolid lip glosses have been produced but it has proved difficult to disperse sufficient pigment to obtain the colour intensity required for a lipstick in previously known cosmetic bases.

Some attempts have been made to address this problem in the past and, for example, U.S. Pat. No. 5,747,017, U.S. Pat. No. 6,001,374, U.S. Pat. No. 6,027,739, U.S. Pat. No. 6,190,681 and US 6,982,077 all relate to liquid lipstick compositions. However, they are very different from the compositions of the present invention as they all comprise several ingredients including a copolymer, for example an acrylate copolymer, and a cosmetic pigment in an alcoholic solvent. Alcohol is not beneficial to the skin and several of the compositions described in these documents are said to produce a tingling or stinging sensation when applied to the lips.

The present invention overcomes the problems of the prior art by providing a liquid or semisolid cosmetic composition, particularly a lipstick composition which does not contain inorganic polymers or alcohols.

In some cases, the cosmetic composition may contain other ingredients, for example perfumes or flavours. Suitably, these are natural perfumes and flavourings, for example essential oils such as rose oil, lemon oil or orange oil.

The amounts of such ingredients are small; typically each additional ingredient is present in an amount of ≦2.5% by weight of the composition.

The cosmetic composition may be, for example a lipstick, blusher or eyeshadow composition. However, suitably, the cosmetic composition is a lipstick.

Suitable pigments for use in lipstick compositions are well known to those of skill in the art and include:
D&C Red No 30/CI73360;
FD&C Red No 4/CI14700;
FD&C Yellow No 5/CI19140;
D&C Red No 7/CI15850;
Pigment White 6/CI77891-Titanium Dioxide;
FDA Red Iron Oxide/CI77491;
FDA Red Iron Oxide/CI77491;
or combinations of one or more of the above.

Alternatively, it is possible to use a blend of pigments such as:
Mica, Iron Oxide/CI77491;
Mica, Titanium Dioxide, Tin Oxide/CI77019, 77891.

Other suitable pigments and combinations of pigments are well known to those of skill in the art.

Suitably, the amount of pigment in the composition will be from about 15% to 40% and typically about 25% to 35%.

In a further aspect of the invention, there is provided a process for the preparation of a liquid or semisolid cosmetic composition as described above, the process comprising adding a pigment to a cosmetic base, wherein the amount of pigment is from about 10 to 50% by weight of the cosmetic composition and wherein the process for preparing the cosmetic base comprises:
a. melting a mixture comprising about 4% to 40% by weight of a wax; and about 60% to 96% by weight of a vegetable oil and heating to a temperature of between about 60 and 100° C., more usually about 65 to 80° C.;
b. cooling the blend to about 40 to 55° C., more usually about 45 to 50° C.;
c. allowing the blend to cool further and harden for between 12 and 24 hours (for example overnight);
d. cutting or breaking the solid product of step (c) into small pieces; and
e. agitating and stirring the pieces, optionally with warming to a temperature of not greater than 45° C. until a mixture of liquid or semisolid consistency is obtained.

In step (a), the blend may suitably be kept at the elevated temperature for about 10-30 minutes, depending on the batch size, with batches on the kilo scale being retained at this temperature for about 10 minutes, and larger batches of up to about 1 tonne being maintained at the elevated temperature for about 30 minutes.

In step (b), the blend may be kept at 40-55° C. for about 12-24 hours. Again small batches on the kilo scale (e.g. of about 1-20 kg) will be kept at this temperature for about 12 hours while for larger batches, e.g. up to about a tonne, the time at this temperature will be about 24 hours.

The hardened mixture produced in step (c) is a solid and has the texture of a conventional lipstick composition.

In step (d), the size of the small pieces will depend upon the type of agitating and stirring equipment to be used in step (e). The size should be such that the agitation and stirring can be carried easily and without causing damage to the stirrer. Since the type of stirrer used will depend on the size of the batch, the size of the pieces into which the mixture is broken will also depend on the batch size. Thus, for small batches on the kilo scale (e.g. of about 1-20 kg), the pieces will usually be smaller than for larger batches, e.g. up to about a tonne.

In step (e), the pieces are rapidly agitated and stirred, usually using a mechanical process. In some cases, the stirring is carried out at room temperature (15 to 25° C.) but in others it is necessary to warm the mixture, for example to 25 to 40° C., more usually about 35 to 40° C. It is important that the mixture is not heated above 45° C. as this has been found to prevent the formation of a composition which is liquid or semisolid at room temperature. The mechanical agitation will be continued until the desired texture/consistency is achieved. Typically, this will be from about 10 to 30 minutes, for example about 15 minutes. However, the exact time taken will depend upon the quantity of the mixture which is used, with smaller batches for example of about 1-20 kg typically taken as little as 10 minutes but larger batches, e.g. up to about a tonne, taking about 30 minutes or in some cases longer.

The resultant product may be described as a dispersion or suspension of the wax within the oil. In texture, it may be a viscous liquid or a thick cream, for example a cream of a consistency similar to that of petroleum jelly. It is, however, less oily in texture than petroleum jelly and therefore is more cosmetically acceptable.

In order to prepare the coloured cosmetic composition for application to the skin or lips, a pigment must be added to the cosmetic base. Suitable pigments and quantities are described in detail below. The pigment may be added at any point in the process, for example it may be blended with the final liquid or semisolid composition. Alternatively, it may be added during the mechanical agitation stage.

In a first method, the pigment may be added during the mechanical agitation stage described above. This method is particularly suitable for small batches where all of the cosmetic base produced is converted to a single product.

However, it is often preferable to prepare large amounts of the cosmetic base, for example 50 to about 1000 kg in size and to use the base to manufacture several different products. In this case, an alternative procedure may be employed in which a required amount of the cosmetic base is broken into small pieces the required pigment is added and the mechanical agitation step is repeated, optionally with warming to a temperature of not greater than 45° C., for example to 25 to 40° C. and more usually about 35 to 40° C. As described above, it is important that the temperature does not exceed 45° C. at this stage.

The agitation process can be repeated as many times as required, provided the temperature of the mixture does not exceed 45° C.

As with the process for preparing the cosmetic base, the size of the pieces will be chosen such that they can be easily agitated and stirred by the equipment used without causing damage to that equipment. Since the type of stirring equipment will depend upon the batch size, the size of the pieces into which the mixture is broken may also depend upon the batch size.

If required, other agents may be added with the pigment, for example perfumes or flavours as described above.

As described above, one of the advantages of the cosmetic composition of the present invention is that, because it is in liquid or semisolid form, it can be simply filled into a suitable container and therefore no complex and expensive moulding process is necessary.

Therefore in a further aspect of the invention there is provided a container having a lid and containing a liquid or semisolid cosmetic composition according to the invention.

In some cases, the liquid or semisolid or semisolid cosmetic composition may be applied with the user's own brush or applicator. However, in an alternative embodiment, an applicator is provided with the container. The applicator may be a brush, sponge or spreader or any other type of applicator suitable for applying a liquid or semisolid formulation to the lips or skin. Suitably, in this embodiment, the applicator may be attached to or form part of the lid of the container and be adapted to sit within the container when the lid is closed.

The cosmetic composition can therefore be used from the bottle using the applicator provided. Alternatively application can be by the user's own brush or sponge applicator. The liquid or semisolid cosmetic composition can be applied easily and effectively. Depending on the blend of pigments used in the composition, it can be used as a lipstick and is capable of delivering vibrant colour to the lips. Alternatively, it can be applied to the cheeks as a blusher or to the eye area as an eyeshadow. The composition has a comfortable “feel” on the skin of the user and is not irritating to the skin.

The invention will now be described in greater detail with reference to the following non-limiting examples.

### Example 1—Liquid Lipstick Formulation

The formulation contained the following ingredients:

| | |
|---|---|
| Candelilla wax | 13% |
| Jojoba oil | 54% |
| Pigment White 6/C177891 - titanium dioxide and | 32.5% |
| D&C Red No. 30/173360 (50:50 mix) | |
| orange essential oil | 0.5% |

Candelilla wax (13 g) and the jojoba oil (54 g) were heated to a temperature of about 70° C. to produce a molten mixture. This mixture was held at this temperature for about 15 minutes. The mixture then cooled to about 45 to 50° C., held at this temperature for about 12 hours and then left overnight to cool to room temperature and solidify.

The solid mixture was then cut into small pieces, the orange oil was added and the mixture was mechanically agitated and stirred at a temperature of 35 to 40° C. about 15-30 minutes using a mechanical mixer until a product with a creamy consistency was obtained. This product was allowed to cool to room temperature.

The final product was a semisolid having a creamy consistency and a colour intensity which made it suitable for use as a lipstick rather than a lip gloss. When applied to the skin, users commented that it had a comfortable feel and was not irritating to the skin.

The foregoing description has been given by way of example only and it will be appreciated by a person skilled in the art that modifications can be made without departing from the scope of the present invention.

1. A coloured cosmetic composition for application to the lips or skin, wherein the cosmetic composition is liquid or semisolid at room temperature and comprises:
from about 10% to 50% by weight of a pigment; and
a cosmetic base;
wherein the cosmetic base is a liquid or semisolid at room temperature and comprises:
about 4% to 40% by weight of a wax; and
about 60% to 96% by weight of a vegetable oil.

2. A coloured cosmetic composition as claimed in claim 1 wherein the wax comprises candelilla wax, bees wax, Japan wax, carnauba wax, soy wax and palm wax or mixtures thereof

3. A coloured cosmetic composition as claimed in claim 1 wherein the vegetable oil comprises include jojoba oil, olive oil, sweet almond oil, castor oil, avocado oil, rosehip oil and argan oil or mixtures of two or more of these.

4. A coloured cosmetic composition as claimed in claim 1 wherein the cosmetic base comprises comprises from about 10% to 35% by weight of a wax and about 65% to 90% by weight of a vegetable oil.

5. A coloured cosmetic composition as claimed in claim 4 wherein the cosmetic base comprises from about 15% to 25% by weight of a wax and about 75% to 85% by weight of a vegetable oil.

6. A cosmetic composition as claimed in claim 1, further comprising one or more additional ingredients selected from perfumes, flavours or mixtures thereof, wherein each additional ingredient is present in an amount of ≦2.5% by weight of the composition.

7. A cosmetic composition as claimed in claim 1 which is a lipstick.

8. A cosmetic composition as claimed in claim 1 comprising about 15 to 40% of a pigment.

9. A process for the preparation of a coloured cosmetic composition as claimed in claim 1, the process comprising:
preparing a cosmetic base and adding a pigment to the cosmetic base, wherein the amount of pigment is from about 10 to 50% by weight of the cosmetic composition and wherein
the process for preparing the cosmetic base comprises:
a. melting a mixture comprising about 4% to 40% by weight of a wax; and about 60% to 96% by weight of a vegetable oil and heating to a temperature of between about 60 and 100° C.;
b. cooling the blend to about 40 to 55° C.;
c. allowing the blend to cool further and harden for between 12 and 24 hours;
d. cutting or breaking the solid product of step (c) into small pieces; and
e. agitating and stirring the pieces, optionally with warming to a temperature of not greater than 45° C. until a cosmetic base of liquid or semisolid consistency is obtained.

10. A process as claimed in claim 9 wherein, in step (a), the blend is kept at the elevated temperature for about 10-30 minutes.

11. A process as claimed in claim 9 wherein, in step (b), the blend is kept at 40-55° C. for about 12-24 hours.

12. A process as claimed in claim 9 wherein, in step (e), the stirring is carried out at room temperature (15 to 25° C.).

13. A process as claimed in claim 9 wherein, in step (e), the agitation and stirring is carried out at a temperature of 25 to 40° C.

14. A process as claimed in claim 9, further comprising adding one or more additional ingredients selected from perfumes, flavours or mixtures thereof, wherein each additional ingredient is present in an amount of ≦2.5% by weight of the composition.

15. A process as claimed in claim 9, wherein the pigment is added at step (e).

16. A process as claimed in claim 9 and which further comprises:
f. breaking a selected amount of the product of step (e) into small pieces;
g. adding a pigment to the pieces to form a mixture, wherein the amount of pigment is from about 10 to 50% by weight of the mixture; and
h. agitating and stirring the mixture, optionally with warming to a temperature of not greater than 45° C. until a coloured cosmetic composition of liquid or semisolid consistency is obtained.

17. A process as claimed in claim 16 wherein, in step (h), the agitation and stirring is carried out at a temperature of 25 to 40° C.

18. A process as claimed in claim 16 wherein one or more additional ingredient is added with the pigment, wherein each additional ingredient is selected from perfumes, flavours or mixtures thereof, and wherein each additional ingredient is present in an amount of ≦2.5% by weight of the composition.

19. A container having a lid and containing a liquid or semisolid cosmetic composition according to claim 1.

20. A container as claimed in claim 19 further comprising an applicator attached to or forming part of the lid of the container and adapted to sit within the container when the lid is closed.
